(19) United States
(12) Patent Application Publication
(10) Pub. No.: US 2006/0240038 A1
(43) Pub. Date: Oct. 26, 2006

(54) Cells used as carriers for bacteria

(75) Inventors: Joachim Fensterle, Hoechberg (DE); Werner Goebel, Gerbrunn (DE); Ulf R. Rapp, Wuerzburg (DE); Jochen Stritzker, Wuerzburg (DE); Andreas Schmidt, Zell am Main (DE); Ivatlo Gentschev, Kist (DE); Tamara Potapenko, Wuerzburg (DE)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: ZENTARIS GmbH, Frankfurt (DE)

(21) Appl. No.: 10/559,663

(22) PCT Filed: Jun. 7, 2004
(86) PCT No.: PCT/DE04/01178

§ 371 Date: Jun. 21, 2006

(30) Foreign Application Priority Data
Jun. 6, 2003 (DE) 103 26 187.7

(51) Int. Cl. A61K 39/12

(52) U.S. Cl. 424/204.1

(57) ABSTRACT

The invention relates to the use of a cell, which is charged with a micro-organism that contains foreign DNA, in particular a bacterial micro-organism, to produce a pharmaceutical composition. Preferably, the foreign DNA codes for a defined active agent and the pharmaceutical composition is designed for use in the prophylaxis or treatment of a disease that can be treated with said active agent.

### FIELD OF THE INVENTION

The invention relates to cells infected with bacteria and to the use thereof for producing a pharmaceutical composition, in particular for the treatment of cancer.

### BACKGROUND OF THE INVENTION AND PRIOR ART

Novel approaches to a therapy of previously incurable or inadequately curable disorders include the various possibilities for gene therapy and immunotherapy.

The intention in gene therapy is for a nucleic acid sequence which codes for a desired protein to be transported by suitable carriers into the target tissue, and to penetrate into cells therein and transduce them to express the desired protein. Numerous different technological approaches to gene therapy have been developed and tested. However, viewed overall, the clinical results of this testing of the various approaches have tended to be disappointing overall and in particular for neoplastic diseases. Technical problems are substantially the reason for this. Thus, the carriers of nucleic acid sequences show a target cell specificity which is too low, the number of cells which can be transduced is too low, and the strength and duration of expression of the desired protein is too small for a therapeutic effect.

One established form of immunotherapy is immunization with an antigen, which is called vaccination. After immunization with an antigen, the body produces specific antibodies and/or specific cytotoxic lymphocytes which have prophylactic or therapeutic activity, for example against infectious agents. Various approaches have been attempted for some decades for the treatment by vaccination of previously insufficiently treatable or incurable disorders. At the forefront of this is the therapy of neoplastic diseases by a tumor vaccination. The aim is to bring about through a tumor vaccine an immune response against the tumor, leading to lysis of tumor cells and eventually to elimination of all the tumor tissue. However, no breakthrough in tumor therapy has been achievable as yet with the various tumor vaccines tested to date. A substantial reason derives from the so-called immunotolerance of the tumor host for his tumor. Thus, although it is possible with a large number of immunotherapy approaches to induce relatively well a tumor-specific T-cell response, this frequently does not correlate with the tumoricidal effect (e.g. Thurner et al., J. Exp Med 190:1669-1678 (1999)). Recent findings point to various causes. These include insufficient penetration of the tumor tissue by specific T cells (Mukai et al., Cancer Research 59:5245-5249 (1999)) and/or inactivation of T cells inside the tumor (for example by TFG-β or by expression of negative regulatory markers such as B7-H1 in the tumor tissue or by stimulation of regulatory T cells having an immunosuppressant effect (Review: Bach, Nature Reviews, 3:189-198 (2003)).

Several methods are currently employed in different clinical phases for tumor vaccination and are frequently based on dendritic cells (summarized in Bancherau et al., Cell, 106:271-4 (2001)). The commonest type of immunization with dendritic cells comprises activation of the cells ex vivo, loading (“pulsing”) thereof with antigen (for example purified protein, tumor cell extract or defined peptides) and subsequent administration thereof. Alternatively, methods which include fusion of cells are also used. In this case, for example, irradiated tumor cells are fused to dendritic cells by suitable methods such as an electric field and subsequently administered (Kugler et al., Nat Med 6:332-6 (2000)).

A novel method has been developed, with the aid of recombinant attenuated bacteria such as, for example, salmonellae and listeriae as carriers of selected tumor antigens, to break through this immunotolerance of the patient for his tumor (DE 102 08 653; DE 102 06 325, not yet published). The mechanism by which this immunotolerance can be broken through is not as yet understood in all its details. However, the accumulation, taking place after injection, of bacteria such as, for example, of salmonellae or listeriae in the tumor tissue, and the inflammation caused by these bacteria there, appear to play a substantial part in this. Thus, it is known that i.v. administration of salmonellae may be followed by accumulation of these bacteria in the tumor tissue. However, kinetic studies have shown that only a few bacteria can be found in the tumor tissue at early times after i.v. injection of bacteria, and these are capable of focal growth preferentially in the tumor tissue. Thus, if relatively large quantities of bacteria are observed in the tumor after i.v. injection, they are derived from relatively few precursors (Mei et al., Anticancer Res 22:3261-6 (2002)). However, this is unfavorable for therapeutic use, for example in the sense of gene therapy with salmonellae as gene carriers, because in this case the colonization of the tumor is not uniform; on the contrary, only a few foci with a high bacterial count are produced.

Tumors contain besides the actual tumor cells and the connective tissue a considerable number of leukocytes, in particular of lymphocytes (tumor-infiltrating lymphocytes; TIL) and of macrophages (tumor-associated macrophages; TAM). It is assumed that the tumor localization of leukocytes is influenced by expression products of the tumor cells, in particular by cytokines, endothelins and also by the hypoxia (Sica et al., Int Immunpharmacol, 2: 1045-1054 (2002); Grimshaw et al., Eur J Immunol, 32:2393-2400 (2002)).

The function of the leukocytes localized in the tumor is contradictory. TAM in particular has been demonstrated to have an antitumor (antigen presentation; cytotoxicity; Funada et al., Oncol Rep, 10:309-313 (2003); Nakayama et al., AntiCancer Res 22:4291-4296); Kataki et al., J Lab Clin Med, 140:320-328 (2002)) and a tumor growth-promoting activity (secretion of growth factors; promotion of angiogenesis and of metastasis; Leek and Harris J., Mammary Gland Biol Neoplasia, 7:177-189 (2002); reduced secretion of cytotoxic cytokines such as Il-1 alpha; Il-1beta; IL-6; TNF alpha; Kataki et al., J Lab Clin Med, 140:320-328 (2002)).

Attempts have been made for some time to influence tumor growth by administering cytotoxic lymphocytes, TIL, natural killer cells, macrophages or dendritic cells. The clinical results were, however, contradictory (Faradji et al., Cancer Immunol Immunotherap, 33:319-326 (1991); Montovani et al., Immunology Today, 13:265-270 (1992); Ravaud et al., British J of Cancer, 71:331-336 (1995); Semino et al., Minerva Biotec, 11:311-317, 1999)). It was possible to show experimentally that injection of slightly activated macrophages may lead to a promotion of tumor growth, but injection of highly activated macrophages may lead to an inhibition of tumor growth (Mantovani et al., Immunology Today 13:265-270 (1992)). In this connection, administration of activated macrophages appears to favor tumor localization (Fidler, Adv Pharmacol, 30:271-326 (1974); Chokri et al., Int J Immunol, 1:79-84, (1990)). Nor has injection of leukocytes which had been transduced in vitro with a gene sequence coding for an antitumor protein as yet resulted in any breakthrough clinically in the treatment of tumors (Hege and Roberts, Current Opinion in Biotechnology, 7:629-634 (1996)). However, it was shown during these studies that leukocytes, but also other cells, especially tumor cells, can reach the tumor tissue after i.v. injection (Shao J et al., Drug Deliv 2 (2001)), but that by far the most of the administered cells settle in normal tissues such as lung, spleen and liver (Adams J, Clin Pathol Mol Path 49:256-267 (1996)).

### TECHNICAL PROBLEM OF THE INVENTION

The invention is based on the technical problem of producing means by means of which the target cell localization, especially tumor localization, of microorganisms comprising foreign DNA coding for active substances can be improved.

Perceptions and principles of the invention, and embodiments.

The invention is based on the perception that macrophages or dendritic cells which have been infected, i.e. loaded, with bacteria in vitro transport them after intravenous administration into the tumor tissue, that the amount of bacteria localized in the tumor after i.v. injection of macrophages loaded with bacteria in vitro was distinctly higher than after i.v. injection of a corresponding amount of free bacteria, that even infected heterologous tumor cells accumulate in tumors, and that this effect is maintained even when the infected cells have been inactivated beforehand by irradiation.

If, for example, macrophages were used as carriers for samonellae, ten times more salmonellae were detectable in the tumor tissue 18 hours after i.v. administration of the macrophages loaded with salmonellae than after i.v. injection of a corresponding amount of free salmonellae in two different transgenic tumor models (lung tumor model: Raf transgenic mice, Kerkhoff et al., Cell Growth Differ, 11:185-90 (2000), breast tumor model: Her-2 transgenic mice, Bouchard et al., Cell, 57:931-6 (1989)).

The findings were similar on use of a heterologous tumor line. The tumor cell line 4T1 (ATCC No. CRL-2539) is derived from a tumor of mammary gland tissue of BALB/c mice and was administered, after infection with attenuated listeriae, in the Raf tumor model described (C57BL/6 background). The finding in this case on use of infected cells was also of a greatly increased number of bacteria in the tumor tissue, which was maintained even on previous irradiation of the cells.

It is thus possible in principle to extend these surprising observations to any cells as long as these cells can be infected by bacteria or onto which bacteria adhere firmly, and thus are carriers of these bacteria. Thus, for example in the abovementioned tumor models, it was found that the localization of salmonellae in the tumor tissue was far greater after i.v. injection of tumor cells infected with salmonellae than after i.v. injection of a corresponding amount of free salmonellae.

Bacteria have a strong adjuvant effect in particular through bacterial constituents such as lipopolysaccharides (LPS), cell wall constituents, flagella, bacterial DNA having immunostimulatory CpG motifs, all of which interact with various so-called Toll-like receptors (TLR) on antigen-presenting cells and are thus able to stimulate them. It is therefore to be expected that infection of cells with bacteria and administration of these cells not only brings about an improved accumulation of the bacteria in the tumor, but that this infection will also result in an inflammation and a strengthening of the systemic and local immune response. This method can thus also be employed for increasing the local immune response as part of an immunotherapy.

The invention thus relates to cells of a mammal which are loaded with bacteria and to the use of these cells for the prevention or treatment of a disorder.

Cells in the context of this invention may be for example autologous, allogeneic or xenogenic macrophages, lymphocytes, dendritic cells or tumor cells. When tumor cells are used they are preferably irradiated or treated with a cytostatic in such a way that their ability to divide is blocked. Such cells are preferably isolated from the blood or from tumors by methods known to the skilled worker. However, it is also possible to use autologous, allogeneic or xenogeneic cells established in culture, called cell lines from normal tissues or from tumors. Such cell lines are obtainable in any type and number for example from cell libraries such as the American tissue cell library (ATCC). It is also furthermore possible to use cells which have been modified by methods known to the skilled worker. Modifications here include in particular genetic modifications, but also additional loading of the cells such as, for example, with peptides, proteins, pharmacological active substances or viral particles.

Loading in the context of the invention is the absorption of bacteria onto the cell, phagocytosis of the bacteria by the cell and/or infection of the cell.

Bacteria in the context of the invention are, for example, Gram-negative and Gram-positive bacteria, preferably optionally intracellular bacteria, preferably salmonellae or listeriae, preferably bacteria which are able to divide but have no pathogenicity for the recipient or whose virulence is attenuated or which are killed. Bacteria whose virulence is attenuated are characterized for example in that in at least one chromosome of these bacteria at least one gene for a metabolic enzyme is deleted or mutated so that the metabolic enzyme is defective. In these bacteria it is possible for i) one gene for an enzyme for synthesizing aromatic amino acids to be deleted in the chromosome, for example the aroA gene which codes for the first enzyme in the biosynthesis of aromatic amino acids, so that these bacteria depend for their growth on the presence of aromatic amino acids, ii) the proteins which make the motility of the bacteria possible to be expressed unimpaired, for example for the ability of the iap and actA genes to function to be retained, and iii) the gene trps coding for the tryptophanyl-tRNA synthetase to be deleted in the chromosome, there having been introduction into these bacteria of plasmids, iv) whose replication has been stabilized by a suitable replication origin, for example by ori pAMβ1 (Simon and Chopin, 1988), v) which comprise the trpS gene coding for tryptophanyl-tRNA synthetase, vi) which comprise a gene for an endolysin, for example the lysis gene of the phage A118 (ply 118; Loessner et al., 1995) under the control of a promoter which can be activated in the cytosol of mammalian cells, for example the actA promoter (PactA, Dietrich et al., 1998), and vii) which comprise at least one nucleotide sequence coding for at least one active substance under the control of a promoter which can be activated in bacteria or in mammalian cells, it being possible for the activation of the promoter to be non-cell-specific, cell-specific, cell cycle-specific, cell function-specific or dependent on metabolites, medicaments or on the oxygen concentration.

Bacteria of this type exhibit, owing to the loss of at least one gene for an essential metabolic protein, a drastic reduction in their virulence, for example measured by their ability to multiply in vivo, and nevertheless show a considerably increased bactofection, a lysis of the bacteria in the cytosol, a release of the plasmids contained in the bacteria, and a stable expression of the active substance encoded by the plasmid. Such a bacterial microorganism very generally comprises a foreign nucleic acid sequence which codes for an active substance and is optionally under the control of a regulatory nucleic acid sequence, where in the chromosomal DNA of the microorganism a natural nucleic acid sequence of the bacterium which codes for the expression of a bacterial enzyme is either deleted or mutated with the proviso that a translation product derived therefrom is non-functional, and where the microorganism comprises no foreign nucleic acid sequence which codes for the enzyme.

Examples of intracellular bacteria are: Mycobacterium tuberculosis, M. bovis, M bovis strain BCG, BCG substrains, M. avium, M intracellailare, M. africanum, M. kansasii, M. marinum, M. ulcerans, M. avium subspecies paratuberculosis, Nocardia asteroides, other Nocardia species, Legionella pneumophila, other Legionella species, Salmonella typhi, S. typhimurium, other Salmonealla species, Shigella species, Yersinia pestis, Pasteurella haemolytica, Pasteurella multocida, other Pasteurella species, Actinobacillus pleuropneumoniae, Listeria monocytogenes, L. ivanovii, Brucella abortus, other Brucella species, Chlamydia pneumoniae, Chlamydia trachomatis, Chlamydia psittaci and Coxiella burnetii.

Examples of attenuations of salmonellae are: inactivating mutations in a pab gene, a pur gene, an aro gene, asd, a dap gene, in nadA, pncB, galE, pmi, fur, rpsL, ompR, htrA, hemA, cdt, cya, crp, dam, phoP, phoQ, rfc, poxA, galU, metL, metH, mviA, sodC, recA, ssrA, ssrB, sirA, sirB, sirC, inv, hilA, hilC, hilD, rpoE, flgM, tonB or slyA, and combinations thereof. The inactivating mutations of the genes which are listed by way of example for attenuation of salmonellae are familiar to the skilled worker.

The invention further relates to cells which are carriers of bacteria, there having been introduction into these bacteria of nucleic acid sequences which code for a protein, these proteins preferably representing active substances for the prevention or treatment of a disorder.

Such proteins may be for example: antigens of infectious agents such as viruses, bacteria, mycoplasmas, parasites, antigens specific for tumors, in particular proteins encoded by oncogenes, antibodies, epitope-binding fragments of antibodies and fusion proteins comprising at least one epitope-binding fragment of an antibody directed for example against an antigen on a tumor cell, on a lymphocyte such as, for example, a T lymphocyte or on an endothelial cell such as, for example, a tumor endothelial cell, enzymes, in particular enzymes for activating inactive precursors of a medicament such as, for example, a β-glucuronidase, a phosphatase, a hydrolase, a lipase, immunospuppressant cytokines such as, for example, IL-10, immunostimulating cytokines such as, for example, IL-1, IL-2, Il-3 or IL-6, chemokines, interferons, growth factors such as, for example, G-CSF, GM-CSF, M-CSF, FGF; VEGF or EGF, or inhibitory proteins for cytokines, chemokines, interferons or growth factors.

The expression of these genes in the bacteria is regulated by suitable promoters, it being possible for these to derive from the bacteria or from viruses or from eukaryotes and to be nonspecifically, cell-specifically or function-specifically activatable.

In a further preferred embodiment of the invention, nucleic acid sequences which enable transmembrane expression or secretion of the gene-encoded protein by the bacterium are attached to the gene. Examples of such so-called signal sequences are described in the references EP 1042495, EP 1015023 and Hess et al., PNAS USA 93:1458-1463 (1996).

The invention further relates to the use of a cell of the invention for the prevention or treatment of a disorder. The cells of the invention are preferably used for treating a neoplastic disease or an immune disease. For this purpose, the gene introduced into the bacteria encodes a protein which i) is tumor-cytolytic, ii) has proinflammatory effects, iii) inhibits negatively regulating immune cells, such as, for example, through inhibition of CTLA-4, of B7-H1 or of CD25 or of TGFβ, iv) has immunosuppressant effects or v) can convert an inactive precursor of a cytotoxic, immunomodulating or immunosuppressant substance into an active substance.

For the prevention or treatment of a disorder, preferably from 100 to 10ˆ9 cells which preferably carry about 0.1 (statistical mean) to 100 bacteria per cell are administered. Such cells are administered locally on the skin, into the circulation, into a body cavity, into a tissue, into an organ or orally, rectally or bronchially at least once.

Disorders for which the cells of the invention are used are, for example, neoplastic diseases, autoimmune diseases, chronic inflammations and organ transplants.

The invention is explained in more detail below by means of exemplary embodiments.

### EXAMPLE TO ILLUSTRATE THE INVENTION

### Example 1

### Provision of Salmonella typhimurium 7207 by Infected Autologous Bone Marrow Macrophages

1.1: Isolation of Bone Marrow Macrophages (MΦ).

BxB23 mice abut 2-3 months old, or MMTV/neu transgenic mice about 2 months old were used to isolate bone marrow macrophages. The macrophages were isolated according to the following protocol: i) remove the femur from the mouse, ii) remove soft tissues from bone in a Petri dish and cut open bilaterally, iii) rinse bone marrow with 2 ml of DMEM 10 (DMEM Gibco with 10% FCS Gibo, 2 mM L-glutamine Gibco, 50 μM β-mercaptoethanol Gibco) with the aid of a syringe in Bluecap with DMEM 10, iv) centrifugation at 1200 rpm for 5′, aspirate and take up in 5 ml of differentiation medium. Adjust to a cell count of 1×10ˆ5 cells/ml in differentiation medium (DMEM 10+10 ng/ml GM-CSF (recombinant mouse granulocyte macrophage colony stimulating factor; RD Systems, Wiesbaden Cat. No.: 415-ML) and distribute in 5 ml portions in Nunc culture dishes (NUNCLON™, 58 mm, NUNC No.: 16955), v) incubate at 37° C. and 10% CO2 for 8 days, vi)

1.2: Infection of Macrophages with Salmonella typhimurium 7207 (SL7207) in vitro.

The MΦ adhering to the NUNC cell culture dish were washed with DMEM and then the adherent cells were harvested with a cell scraper, counted and taken up in differentiation medium. Infection with SL7207 (Hoiseth S. K. et al., Nature 291:238-239 (1981)) took place according to the following protocol: i) 37° C., 1 h in an incubator: MOI (multiplicity of infection) 1:20, ii) 10ˆ6 macrophages were seeded in 2 ml of medium in a NUNC culture dish and incubated with 2×10ˆ7 bacteria (MOI=20) at 37° C. for 1 h, iii) then wash, iv) incubate with gentamycin (final conc. 100 μg/ml (Sigma)) 1 h, 37° C., v) wash, determine cell count, plate out on brain heart infusion (BHI) plates (Gibco) for counting the bacterial colony-forming units (CFUs).

1.3: Results of the Loading of Macrophages.

With an MOI of 20 and a loading time of one hour it is possible constantly to detect about 10ˆ4 salmonellae in 10ˆ5 macrophages. The loading density remained approximately constant for 12 hours after the loading and shows no bacterial proliferation at all.

1.4: Administration of Macrophages Infected “in vitro” with SL 7207 in BxB23 and MMTV/neu Tumor Mice

5×15ˆ5 bone marrow macrophages infected in vitro and suspended in 100 82 l of PBS were injected i.v. and per mouse into the tail vein of BxB23 and MMTV/neu tumor mice (the experimental animals used showed advanced tumor development, age about 12 months, the lung mass due to the lung tumors in the BxB23 mice amounted to 0.75-1.25 g). Depending on the experiment, a bacterial count of 3-5×10ˆ4 S. typhimurium 7207 was injected (determined by counting the CFUs) per mouse. As control, S. typhimurium 7207 was administered i.v. (2.5×10ˆ5 bacteria suspended in 100 μl of PBS per mouse) to BxB23 and MMTV/neu tumor mice. After 18 h, the animals were sacrificed and the CFU (plated out on BHI plates) in the lung (BxB23) and in the tumor (MMTV) were determined. The progress of the infection was investigated in the control group after i.v. injection of S. typhimurium aroA 7207. For this purpose, the bacterial count was investigated by determining the CFUs at various times using the same protocol.

1.5: Accumulation of S. typhimurium 7207 in Tumors After i.v. Injection:

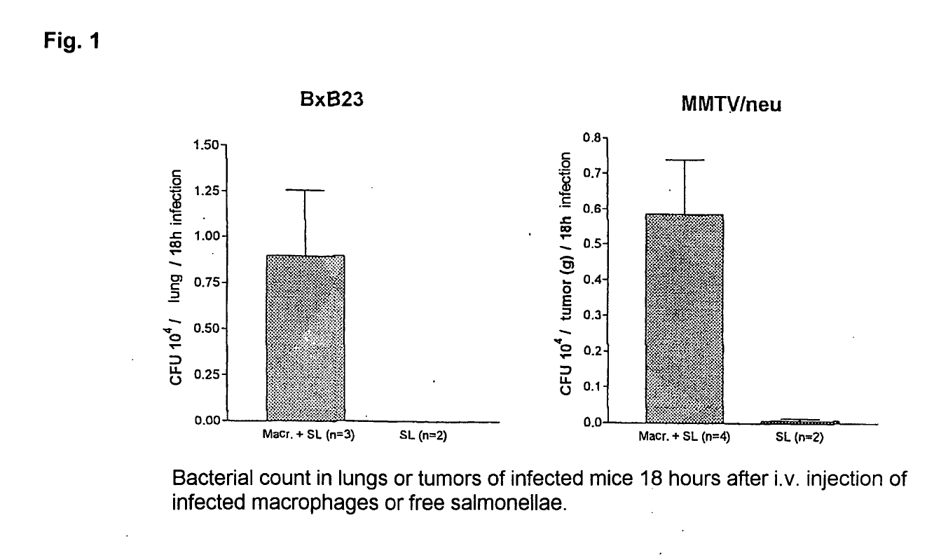

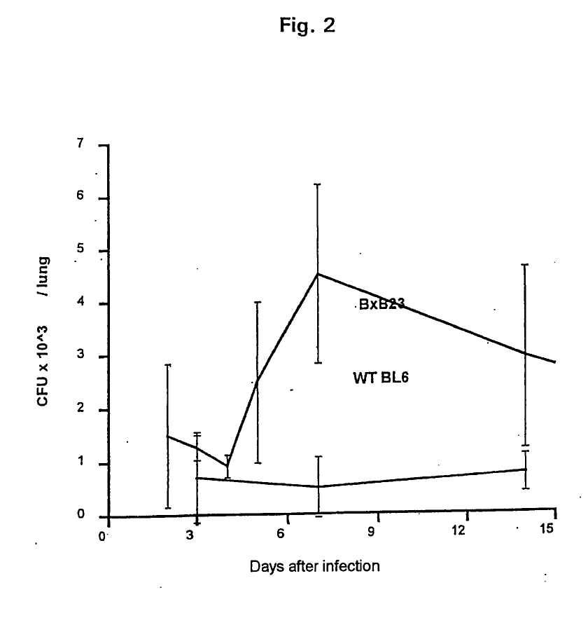

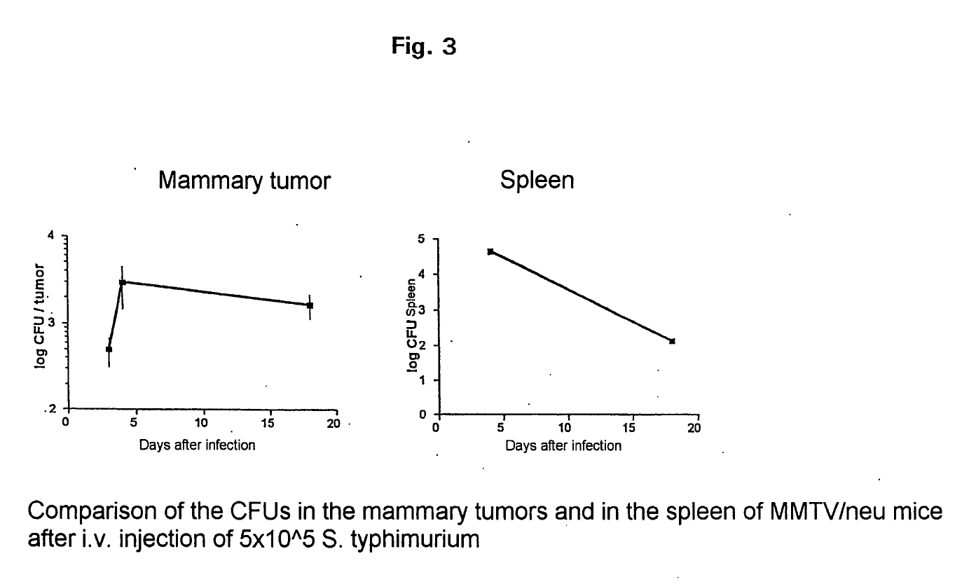

The amount of salmonellae detected in the tumor-bearing lungs of BxB23 mice and in mammary tumors of MMTV/neu mice after administration of salmonellae-infected macrophages was more than ten times that in animals in the control group, which had been treated with free salmonellae, 18 hours after the infection. Following injection of bacteria-loaded macrophages, the accumulation of the bacteria even 18 hours after the injection was as high (factor 10 higher than on injection of naked bacteria, see FIG. 1 and relevant table) as could be achieved comparatively in the control group (i.e. after injection of the bacteria alone) only after some days (day 5 after infection). It was accordingly possible with the aid of the bacteria-loaded cells of the invention to accumulate a distinctly larger number of bacteria in a substantially shorter time in the tumor than was possible after injection of the pure bacterial suspension. Determination of the CFUs in the lungs and the organs on days 1, 7, 14 after injection of the cells of the invention revealed that the CFUs remained at a constant high level or increased in the lungs of the tumor-bearing BxB23 mice, whereas the CFUs in the lungs of the C57BL/6 control mice and in the spleens of the BXB23 mice and of the C57BL/6 mice fell distinctly below the values in the respective lungs or were no longer detectable (see FIG. 2 and FIG. 3, and relevant tables; FIG. 2 shows a comparison of the CFUs in lungs of (lung) tumor-bearing BxB23 mice with lungs of the C57BL/6 control animals, FIG. 3 a comparison of the CFUs in the mammary tumors and in the spleen of MMTV/neu mice after i.v. injection of 5×105 S. typhimurium).

### Example 2

### Provision of L. monocytogenes by Infected Heterologous Cells

4T1 cells (ATCC CRL-2539) of a tumor line from a mammary gland tumor of BALB/c mice were infected with the attenuated L. monocytogenes strain and an MOI of 10 over a period of 1 h. The cells were then washed, and free bacteria were killed by incubation in the presence of gentamycin for one hour. Determination of the CFUs revealed a loading of the cells with 0.15 bacteria per cell. The cell count was adjusted to 5×10ˆ6 cells per ml in PBS. In addition, some of the infected cells were inactivated by irradiation. 0.1 ml of this suspension [i.e. 5×10ˆ5 infected cells (measured CFU of listeriae: 7.3×10ˆ4), 3.5×10ˆ5 infected and irradiated cells or (counted CFUs) 3.5×10ˆ5 free listeriae, each in 100 μl of PBS] per mouse were injected i.v. into tumor-bearing BxB23 mice (age >10 months) or C57BL/6 mice of the same age.

With the radiation dose used there is a reduction, detected by CFU determination, in free bacteria by a maximum of 25%, resulting in a calculated infectious dose of about 3.8×10ˆ4 bacteria in the case of irradiated cells.

17 h after the infection, the bacterial CFUs were determined in the lung and spleen by serial plating on BHI plates (Gibco) (limit of detection 10 bacteria per organ).

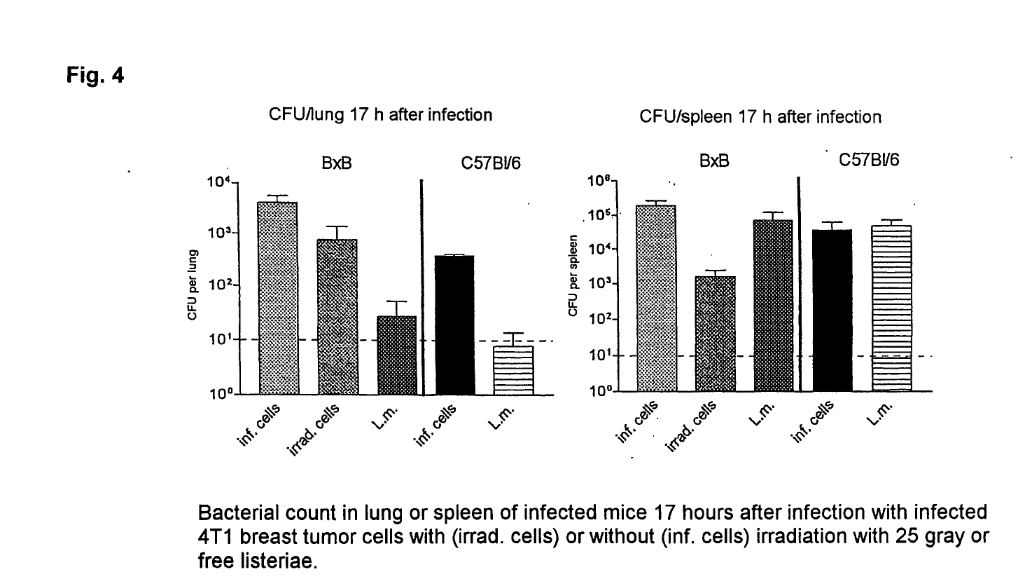

All animals showed a successful infection according to the detectable CFUs in the spleen. The number of CFUs after injection of the living or irradiated cells of the invention was distinctly higher in the lungs of the tumor-bearing BxB23 mice and of the C57BL/6 control mice than after injection of the bacterial suspension, and the bacterial count after injection of the cells of the invention was distinctly increased in the lungs of the tumor-bearing BxB23 mice compared with the bacterial count in the lungs of the C57BL/6 control mice (factor 10). Considerably more bacterial CFUs were detectable in the spleen of all groups than in the lung, but it was not possible to detect a clear difference in the number of bacterial CFUs after injection of the cells of the invention or of the bacterial suspension both in tumor-bearing BxB23 mice and in the C57BL/6 control mice (see FIG. 4 and the relevant table).

As already demonstrated in the abovementioned control groups in the experiments with macrophages loaded with virulence-attenuated S. typhimurium 7207, in the case of virulence-attenuated L. monocytogenes too there is a reduction in the number of the bacterial CFUs in the spleen and in other, nontumor-bearing organs within a period of about 5 days, but a maximum of 14 days after injection both of the cells of the invention and of the pure bacterial suspension, both in the tumor-bearing BxB23 mice and in C57BL/6 control mice to levels which are distinctly below the levels of the CFUs in the lungs of the (lung) tumor-bearing BxB23.

In contrast thereto, the initially increased number of bacterial CFUs in the lungs of the (lung) tumor-bearing BxB23 mice at least persists after injection of the cells of the invention over the entire period or even increases initially, only to fall again after a prolonged plateau phase.

TABLE 1
Bacterial count in lungs or tumors of infected
mice 18 hours after i.v. injection of infected
macrophages or free salmonellae.

| | S.T. + macrophages | | | S. typhimurium | | |
|---|---|---|---|---|---|---|
| Mouse line | CFU | SEM | n | CFU | SEM | n |
| BxB23 | 9.000 | 3.600 | 3 | 0 | 0 | 2 |
| (lung) | | | | | | |
| MMTV Her | 5.850 | 1.552 | 4 | 66 | 66 | 2 |
| (tumor) | | | | | | |

TABLE 2
Comparison of the CFUs in lungs of (lung) tumor-bearing
BxB23 mice with lungs with the C57BL/6 control animals

| | BxB23 | | | WT C57/B16 | | |
|---|---|---|---|---|---|---|
| Day | CFU | SEM | n | CFU | SEM | n |
| 2 | 1.485 | 1.335 | 2 | | | |
| 3 | 1.255 | 229 | 7 | 700 | 600 | 2 |
| 4 | 910 | 210 | 2 | | | |
| 5 | 2.469 | 1.503 | 6 | | | |
| 7 | 4.499 | 1.694 | 6 | 500 | 400 | 2 |
| 14 | 2.900 | 1.700 | 2 | 750 | 250 | 2 |
| 18 | 2.225 | 975 | 2 | | | |

TABLE 3
Comparison of the CFUs in the mammary tumors
and in the spleen of MMTV/neu mice after i.v. injection
of 5 × 10⁵ S. typhimiuium aroA

| | Tumor | | | Spleen | | |
|---|---|---|---|---|---|---|
| Day | Log (CFU) | SEM | n | Log (CFU) | SEM | n |
| 3 | 2.67 | 0.24 | 2 | 4.65 | 0.10 | 2 |
| 4 | 3.19 | 0.68 | 4 | | | |
| 18 | 3.20 | 0.20 | 2 | 2.14 | 0.06 | 2 |

TABLE 4
Bacterial count in infected mice 17 hours
after infection with infected 4T1 breast tumor cells
with (irrad. cells) or without (inf. cells) irradiation
with 25 gray or free listeriae.

| | BxB23 | | | C57BL/6 | |
|---|---|---|---|---|---|
| | Inf. | Inf. irradiated | L. mon. | Inf. | L. mon. |
| | cells | cells | aroA | cells | aroA |
| Log | 3.572 | 2.437 | 0.6344 | 2.601 | 0.4337 |
| (CFU) | | | | | |
| SEM | 0.1333 | 0.5261 | 0.6344 | 0.01688 | 0.4337 |
| n | 3 | 3 | 3 | 3 | 3 |

TABLE 5
Bacterial count in the spleen of infected mice
17 hours after infection with infected 4T1 breast tumor
cells with (irrad. cells) or without (inf. cells)
irradiation with 25 gray or free listeriae.

| | BxB23 | | | C57BL/6 | |
|---|---|---|---|---|---|
| | Inf. | Inf. irradiated | L. mon. | Inf. | L. mon. |
| | cells | cells | aroA | cells | aroA |
| Log | 5.224 | 2.934 | 4.045 | 4.37 | 4.57 |
| (CFU) | | | | | |
| SEM | 0.1596 | 0.4338 | 0.9131 | 0.3023 | 0.2573 |
| n | 3 | 3 | 3 | 3 | 3 |

1. A mammalian cell which is loaded with
a bacteria for the prophylaxis or therapy of a disorder,
wherein the cell is autologous, allogeneic or xenogeneic and is selected from the group consisting of macrophages, dentritic cells, granulocytes, lymphocytes, tumor cells and tissue cells.

2. The mammalian cell as claimed in claim 1, which is inactivated by irradiation or other methods.

3. The mammalian cell as claimed in claim 1, wherein the bacteria are alive, nonvirulent, virulence-attenuated, or dead.

4. The mammalian cell as claimed in claim 1, wherein the bacteria are selected from the group consisting of Mycobacterium tuberculosis, M. bovis, M. bovis strain BCG, BCG substrains, M. avium, M. intracellailare, M. africanum, M. kansasil, M. marinum, M. ulcerans, M. avium subspecies paratuberculosis, Norcardia asteroides, other Nocardia species, Legionella pneumophila, other Legionella species, Salmonella typhi, S. typhimurium, other Salmonella species, Shigella species, Yersinia pestis, Pasteurella haemolytica, Pasteurella multocida, other Pasteurella species, Actinobacillus pleuropneumoniae, Listeria monocytogenes, L. ivanovii, Brucella abortus, other Brucella species, Chlamydia pneumoniae, Chlamydia trachomatis, Chlamydia psittaci, and Coxiella burnetii.

5. The mammalian cell as claimed in claim 1, wherein the bacteria harbor recombinant DNA that encodes at least one active substance.

6. The mammalian cell as claimed in claim 5, wherein at least one active substance is produced by the bacteria with the aid of suitable promoters, or the expression thereof is under the control of a eukaryotic promoter.

7. The mammalian cell as claimed in any claim 1, wherein the bacteria produces an active substance that localizes intracellularly, that is associated with a membrane of the bacteria, or that is secreted.

8. The mammalian cell as claimed in any claim 1, wherein the active substance is selected from the group consisting of antigens of infectious agents, antigens specific for tumors, antibodies, epitope-binding fragments of antibodies, fusion proteins enzymes, imunospuppressant cytokines, immunostimulating cytokines, growth factors and inhibitory proteins.

9. A method for the prophylaxis or therapy of a disorder, comprising:
administrating an effective amount of a mammalian cell of claim 1 to a subject, wherein the active substance and/or vaccine antigen produced by the bacteria blocks negative regulatory elements in a tumor tissue.

10. The method of claim 9, wherein the bacteria serve as a proinflammatory stimulant in a tumor tissue.

11. The method of claim 9, wherein dendritic cells or macrophages are employed simultaneously as a carrier for the vaccine antigen.

12. The method of claim 9, wherein the active substance and/or the vaccine antigen is loaded ex vivo onto dentritic cells or onto macrophages.

13. The method of claim 12, wherein the vaccine antigen comprises defined peptides.

14. The method of claim 9, wherein the mammalian cell is fused to another cell which expresses a tissue antigen or a tumor antigen.

15. The method of claim 14, wherein the fused cells are autologous tumor cells.

16. (canceled)

17. The method of claim 9, wherein the bacteria is a microorganism that comprises a foreign DNA, for producing a pharmaceutical composition.

18. The method of claim 17, wherein the foreign DNA codes for a defined active substance, and wherein a pharmaceutical composition is intended for the prophylaxis or treatment of a disorder which can be prevented and/or treated with the active substance.

19. The mammalian cell of claim 8, wherein the infectious agent is a virus, a bacteria, a mycoplasma, or a parasite.

20. The mammalian cell of claim 8, wherein the enzyme is an enzyme for activating inactive precursors of a medicament.

21. The mammalian cell of claim 20, wherein the enzyme for activating inactive precursors of a medicament is a β-glucuronidase, a phosphatase, a hydrolase, or a lipase.

22. The mammalian cell of claim 8, wherein the imunospuppressant cytokine is IL-10.

23. The mammalian cell of claim 8, wherein the immunostimulating cytokine is IL-1, IL-2, IL-3, IL-6, a chemokine, or an interferon.

24. The mammalian cell of claim 8, wherein the growth factor is G-CSF, GM-CSF, M-CSF, FGF, VEGF, or EGF.

25. The mammalian cell of claim 8, wherein the inhibitory protein is specific for a cytokine, a chemokine, an interferon, or a growth factor.

26. The mammalian cell of claim 8, wherein the fusion protein comprises at least one epitope-binding fragment of an antibody directed against an antigen on a tumor cell, a lymphocyte, or an endothelial cell.

27. The mammalian cell of claim 26, wherein the lymphocyte is a T lymphocyte.

28. The mammalian cell of claim 26, wherein the endothelial cell is a tumor endothelial cell.
